(19) United States
(12) Patent Application Publication
(10) Pub. No.: US 2009/0215874 A1
(43) Pub. Date: Aug. 27, 2009

(54) APTAMERS AS AGONISTS

(75) Inventors: Bruce A. Sullenger, Durham, NC (US); James McNamara, Durham, NC (US); Eli Gilboa, Durham, NC (US)

Correspondence Address: NIXON & VANDERHYE, PC, 901 NORTH GLEBE ROAD, 11TH FLOOR, ARLINGTON, VA, 22203, US

(73) Assignee: DUKE UNIVERSITY, Durham, NC (US)

(21) Appl. No.: 12/066,598

(22) PCT Filed: Sep. 15, 2006
(86) PCT No.: PCT/US06/36090

§ 371 Date: Oct. 2, 2008

Related U.S. Application Data
(60) Provisional application No. 60/716,976, filed on Sep. 15, 2005.

(51) Int. Cl. A61K 31/7088; C07H 21/04; A61P 35/00

(52) U.S. Cl. 514/44 R; 536/23.1

(57) ABSTRACT

The present invention relates, in general, to aptamers and, in particular, to aptamers capable of stimulating target molecules and to methods of using same.

This application claims priority from U.S. Provisional Application No. 60/716,976 filed Sep. 15, 2005, the entire content of which is incorporated herein by reference.

### TECHNICAL FIELD

The present invention relates, in general, to aptamers and, in particular, to aptamers capable of stimulating target molecules and to methods of using same.

### BACKGROUND

Antibodies that stimulate various cell-surface receptors have been described by a number of groups. Some of these stimulatory antibodies have important clinical applications. Such antibodies generally stimulate their target receptors by bringing two receptor proteins into close proximity of one another. They are able to “cross-link” their targets because they contain two target-binding domains per antibody molecule.

Stimulation of T cells results in a number of intracellular signaling events that lead to enhanced cellular proliferation and cytokine secretion. Maximal stimulation of T cells requires the activation of two types of receptors: the T cell receptor and an additional co-stimulatory receptor that can be one of a number of different receptors expressed on the T cell surface, including 4-1BB. Suboptimal stimulation of the T cell receptor with an anti-CD3e antibody induces the expression of 4-1BB on the cell surface. 4-1BB can then be stimulated with 4-1BBL, its natural ligand, which is expressed on the surface of dendritic cells. Antibodies that bind 4-1BB have been shown to stimulate this receptor in vitro. When administered to animals bearing tumors, these antibodies generally enhance the immune response to the cancer cell, in some cases resulting in complete clearance of the tumors.

The present invention provides a novel approach to stimulating target molecules, including cell-surface receptors. In accordance with the instant invention, nucleic acid aptamers are used to effect stimulation.

### SUMMARY OF THE INVENTION

The present invention relates generally to aptamers. More specifically, the invention relates to aptamers that can function as agonists and to methods of using same.

Objects and advantages of the present invention will be clear from the description that follows.

### BRIEF DESCRIPTION OF THE DRAWINGS

FIG. 1. SELEX.

FIGS. 2A-2C. Identification of RNA aptamers with high affinity for mouse 4-1BB. FIG. 2A. Binding of Sel I to M4-1BB. FIG. 2B. Binding of Sel I and selection rounds to M4-1BB in 150 mM NaCl. FIG. 2C. M4-1BB Selex/Rnd 12 Clones.

FIG. 3. Strategy for stimulating 4-1BB in vitro.

FIG. 4. Interferon-γ ELISA with supernatants of CD8+ T cell cultures.

FIGS. 5A-5G. CFSE proliferation assay with CD8+ T-cells. FIG. 5A. Day 2. Untreated. FIG. 5B. Day 4, +Hams IgG. FIG. 5C. Day 4, +anti-CD3, +RlgG2a, FIG. 5D. Day 4, +anti CD3, and anti-4-1BB, FIG. 5E. Day 4, anti-CD3, +M12-12, FIG. 5F. Day 4, +anti-CD3, +mut M12-12, FIG. 5G. Day 4, +Hams IgG, +M12-12.

FIGS. 6A-6E. FIG. 6A. 40 bp randomized regions of round 12 of M4-1BB selex. FIG. 6B. 40 bp randomized regions of round 10 of M4-1BB selex. FIG. 6C. 40 bp randomized regions of round 12 of Toggle 4-1BB selex. FIG. 6D. 40 bp randomized regions of round 10 of Toggle 4-1BB selex. FIG. 6E Sequences flanking the 5′ ends of full length aptamers.

FIG. 7. Interferon γ ELISA.

### DETAILED DESCRIPTION OF THE INVENTION

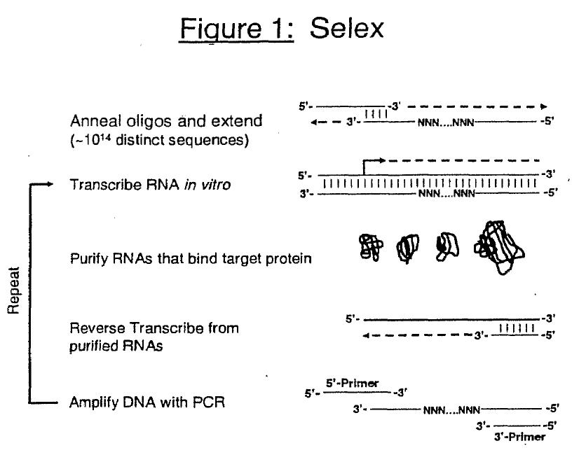

The present invention results from the demonstration that nucleic acids aptamers can be engineered to stimulate target molecules. The aptamers of the invention can be selected for a particular target (e.g., receptor) using the SELEX procedure (FIG. 1) (see, for example, U.S. Pat. Nos. 5,475,096 and 5,270,163 and WO 91/19813). The bases of the RNA used in the selections can be modified (e.g., 2′-fluoro modified) in order to increase stability.

The invention is exemplified below with reference to 4-1BB, an inducible, co-stimulatory receptor of T-cells. The invention, however is not limited to RNA aptamers to 4-1BB but rather encompasses RNA aptamers that stimulate other target molecules, including other receptors (e.g., T cell receptors). Depending on the target sought to be stimulated, the aptamers can be monomeric or they can be multimerized using any of a variety of approaches, including multimerization on solid supports (e.g. beads) as described in the Examples that follow.

The aptamers of the invention, capable of stimulating target molecules, can be used in lieu of stimulatory antibodies and recombinant proteins in a variety of therapeutic settings. 4-1BB, for example is a promising therapeutic target for cancer immunotherapy and various autoimmune diseases. The multimerized aptamers described herein, for example, are contemplated for use in inhibiting tumor growth.

The aptamers of this invention can be formulated into compositions using methods well known in the art. Appropriate carriers can be selected, depending, for example, the aptamer, the target molecule, and the effect sought. Optimum dosing regimens can be readily established by one skilled in the art.

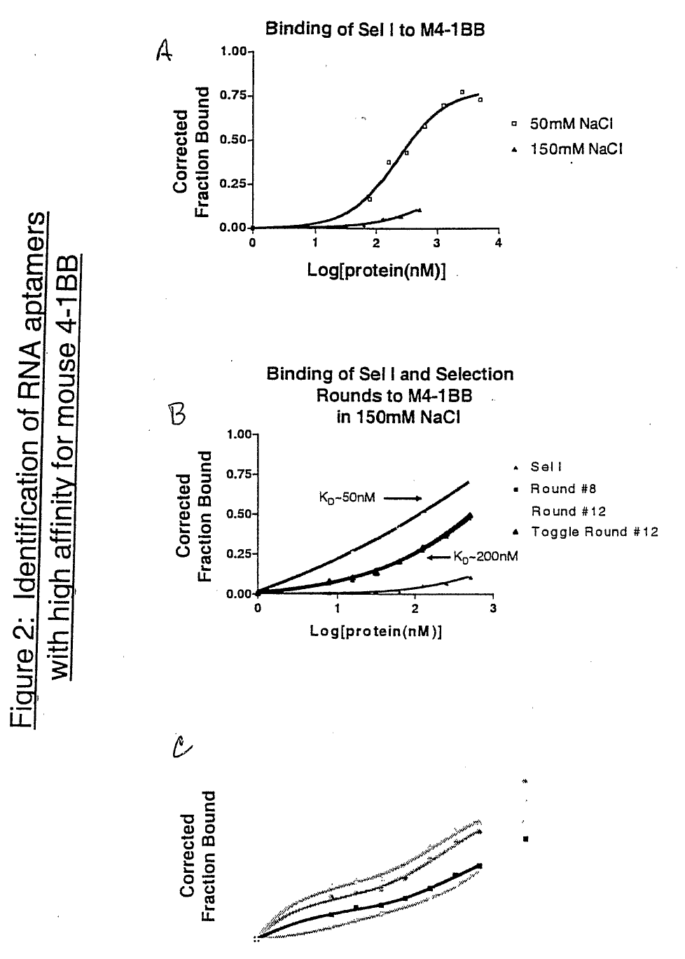

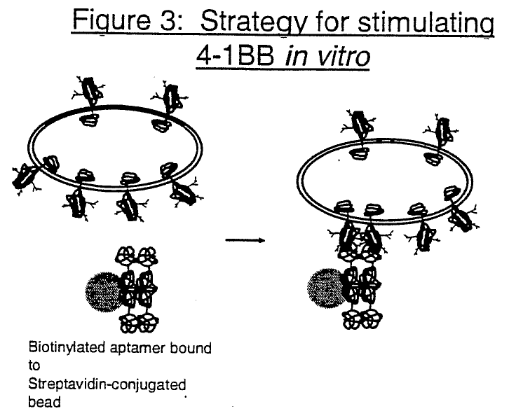

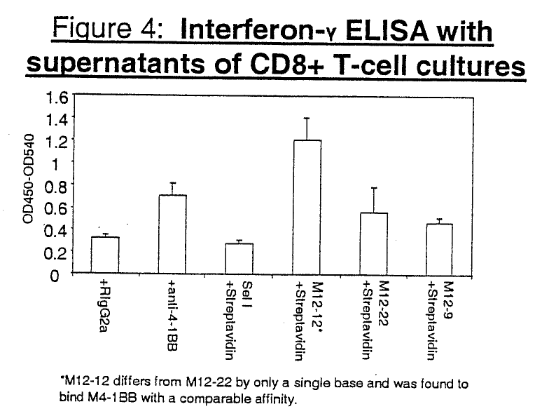

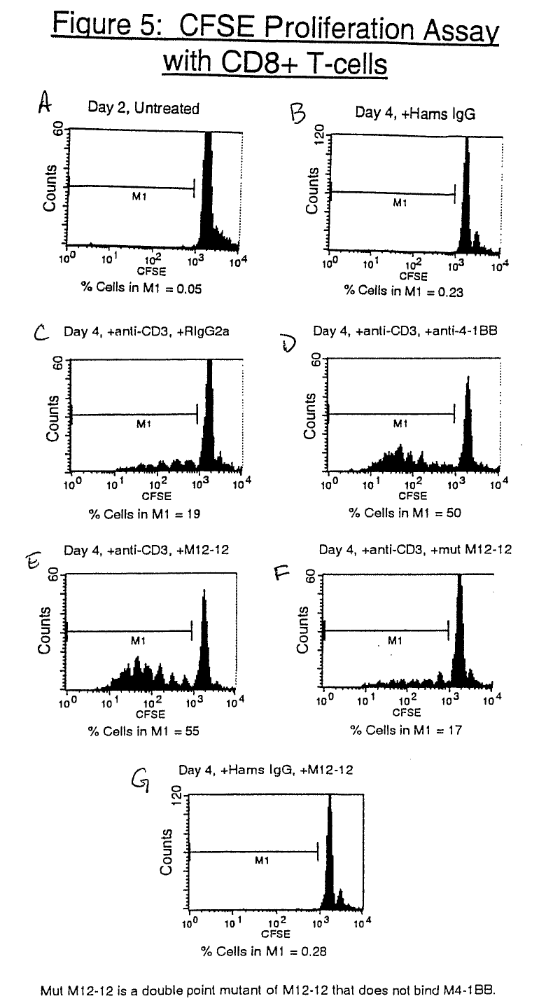

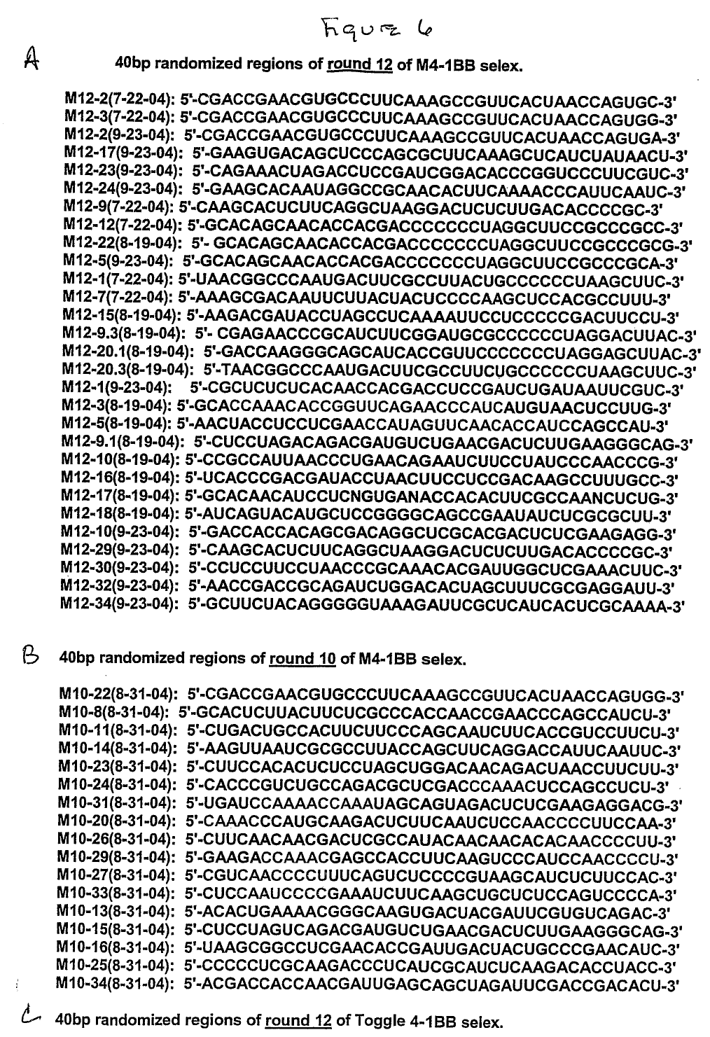

Using the SELEX procedure described in FIG. 1, a number of RNA aptamers that bind with high affinity (Kd's<50 nM) to the extracellular portion of the mouse and human 4-1BB proteins were identified (see FIG. 2). These aptamers were screened for their ability to induce mouse CD8+ T cells to proliferate and secrete interferon-γ. For these screens the aptamers were multimerized on the surface of beads that were then incubated with the cells (see FIG. 3). A subset of the high-affinity binders was found to induce both cellular proliferation and interferon-γ secretion (see FIGS. 4 and 5).

Certain aspects of the invention can be described in greater detail in the non-limiting Example that follows. (See also U.S. Published Appln. Nos. 20030083294 and 20030175703.)

### EXAMPLE 1

RNA aptamers were selected to the T cell co-stimulatory receptor 4-1BB (CD 137) using the SELEX procedure. The pyrimidines in the RNA used in these selections were 2′-fluoro modified in order to protect the RNAs from extracellular RNAses and thus make them suitable for animal studies or therapeutics.

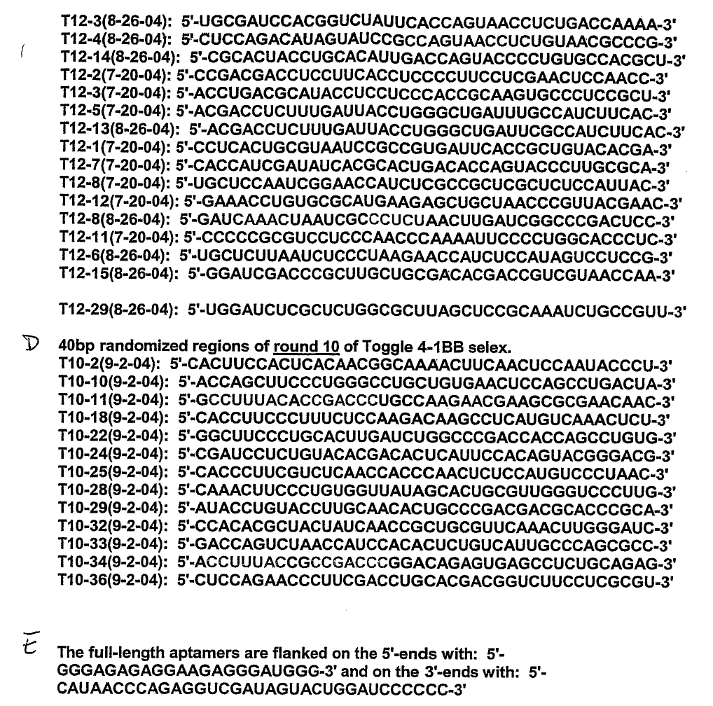

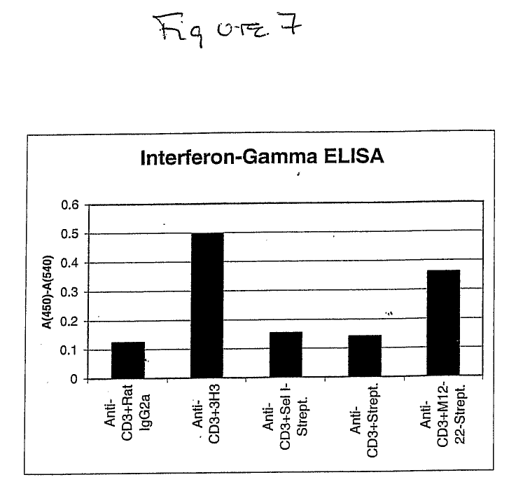

Three selections were carried out for high-affinity RNA aptamers to 4-1BB. The first selection was carried out with a fusion protein of the extracellular portion of mouse 4-1BB and the fixed portion of human IgGl (Fc) using an RNA library with 40 randomized bases. A total of 12 rounds of selection were completed. The round 12 pool of aptamers bind m4-1BB with a dissociation constant of approximately 50 nM. The second selection was carried out with fusion proteins of the extracellular portions of both mouse and human 4-1BB fused with Fc; six rounds were carried out with the mouse 4-1BB fusion followed by two rounds with the human 4-1BB fusion and then four additional rounds alternating each round between mouse and human 4-1BB isoforms. This second selection was also carried out with an RNA library with 40 randomized bases. The pool of aptamers obtained from this selection bind h4-1BB and m4-1BB with dissociation constants of approximately 23 nM and 200 nM, respectively. The third selection was carried out with the human 4-1BB-Fc fusion with an RNA library containing 20 randomized bases. After 9 rounds, the RNA pool obtained from this library binds h4-1BB with a dissociation constant of approximately 20 nM. (See FIG. 6.)

### EXAMPLE 2

Selection of aptamers to mouse 4-1BB yielded a number of sequences that bind m4-1BB with high affinity. These high-affinity binders were tested for their ability to stimulate 4-1BB in vitro. Because aptamers generally bind only one protein per aptamer molecule, aptamers were multimerized in order to cross-link 4-1BB on the cell surface. To multimerize the aptamers, they were labeled on their 5′-ends with biotin and then bound to streptavidin-coated beads. Because each streptavidin protein is able to bind up to four biotin-conjugated molecules, the streptavidin-binding step multimerizes the aptamers on the surface of the beads. Aptamers bound to streptavidin-coated beads were then tested for their ability to stimulate 4-1BB on mouse T cells.

CD8+ T cells were isolated from the spleens of BALB/C mice and then incubated in 96-well round-bottomed dishes at 10⁶ cells per well for 20 hours with a suboptimal concentration of anti-CD3e (1 μg/ml). Then, as a positive control, an anti-4-1BB antibody that is known to stimulate 4-1BB (3H3) was added at 5 μg/ml to some of the wells and, as a negative control, an isotype-matched control antibody (rat IgG2a) was added to other wells at 5 μg/ml. At the same time, 1.25×10⁶ streptavidin-coated magnetic beads that were coupled to either a randomized library of biotinylated RNA sequences or to individual biotinylated aptamers that bind m4-1BB with high affinity (˜50 nM), were added to additional wells of suboptimally stimulated cells. After incubating the cells for an additional 48 hours, an ELISA was carried out to measure relative levels of interferon-γ in the cell supernatants.

The anti-4-1BB antibody (see “Anti-CD3+3H3” in FIG. 7) typically produced a 3-4-fold increase in interferon-γ compared with the isotype-matched control antibody (see “Anti-CD3+Rat IgG 2a” in FIG. 7). The beads coupled to the randomized RNA library (see “Anti-CD3+Sel I Strept.” in FIG. 7) induced a comparable level of interferon-γ as the isotype-matched negative control antibody. Two of the aptamer sequences tested resulted in substantial increases in the interferon-γ levels over the negative controls. The more effective of the two, M12-22 (see “Anti-CD3+M12-22-Strept.” in FIG. 7), induced interferon-gamma levels that were 2.7- to 3-fold greater than that induced by the randomized RNA library. The streptavidin-coated beads alone (see “Anti-CD3+Strept.” in FIG. 7) yielded comparable interferon-γ levels to the other negative controls.

As an additional measure of 4-1BB stimulation, cellular proliferation was also measured in cultures of mouse CD8+ T cells stimulated in the same manner as described above. Approximately a 3-fold increase in proliferation in response to aptamer M12-22 compared with a control was found, double point mutant aptamer. Proliferation in response to M12-22 was comparable to that of the anti-4-1BB antibody positive control while the proliferation in response to the mutant aptamer was comparable to that of the isotype-matched control antibody.

Together, the interferon-γ and proliferation assays indicate that the multimerized M12-22 aptamer can stimulate 4-1BB.

All documents and other information sources cited above are hereby incorporated in their entirety by reference.

What is claimed is:

1. A nucleic acid aptamer that binds to a target molecule with high affinity and stimulates said target molecule.

2. The aptamer according to claim 1 wherein said target molecule is a cell surface receptor.

3. The aptamer according to claim 2 wherein said cell surface receptor is a T-cell surface receptor.

4. The method according to claim 3 wherein said cell surface receptor is 4-1BB.

5. The method according to claim 1 wherein said aptamer is a monomer.

6. The method according to claim 1 wherein said aptamer is a multimer.

7. The method according to claim 6 wherein said multimer is bound to a solid support.

8. The aptamer according to claim 1 wherein at least 1 base of said aptamer is modified.

9. The aptamer according to claim 8 wherein at least 1 base of said aptamer is 2′-fluoro modified.

10. The method according to claim 1 wherein said aptamer is multimerized and is M12-22.

11. A composition comprising said aptamer according to claim 1 and a carrier.

12. A method of stimulating a target molecule comprising contacting said target molecule with a nucleic acid aptamer that binds thereto with high affinity and stimulates the activity thereof.

13. A method of inhibiting growth of a tumor in a patient in need thereof comprising administering to said patient amount of the aptamer according to claim 4 sufficient to effect said inhibition.

14. The method according to claim 13 wherein said aptamer is M12-22.

15. The method according to claim 14 wherein said aptamer is multimerized M12-22.
